(19) United States
(12) Patent Application Publication
(10) Pub. No.: US 2003/0115954 A1
(43) Pub. Date: Jun. 26, 2003

(54) Upper extremity exoskeleton structure and method

(76) Inventors: Vladimir Zemlyakov, Haverhill, MA (US); Patrick McDonough, Kensington, NH (US)

Correspondence Address: Vladimir Zemlyakov, 536 Hadley West Drive, Haverhill, MA, 01832, US

(21) Appl. No.: 10/017,280

(22) Filed: Dec. 7, 2001

(51) Int. Cl. A61B 5/22; G01L 3/24; G01L 5/00; A61F 5/00

(52) U.S. Cl. 73/379.01

(57) ABSTRACT

The adjustable to a user's upper extremity the exoskeleton structure provides testing and exercising of the whole upper extremity in a realistic manner without infringement of a locomotor act and with selective biomechanical information and exercise loading in each anatomical motion direction of every joint simultaneously. The exoskeleton structure comprises jointed means for connection with a user's shouldergirdle, upperarm, forearm, and hand. Those means include measuring-loading blocks to measure muscle forces and joint angles and to apply a dosed exercise load. All measuring-loading blocks in the exoskeleton structure are identical. The exoskeleton structure is able to provide a realistic and comprehensive information about both a complex locomotor act and a selective mono-planar motion for both isometric and isotonic muscular contractions.

### STATEMENT REGARDING FEDERALLY SPONSORED RESEARCH OR DEVELOPMENT

Not applicable

### CROSS REFERENCE TO RELATED APPLICATIONS

Not applicable

### REFERENCE TO A MICROFISH APPENDIX

Not applicable

### BACKGROUND OF THE INVENTION

The present invention relates generally to the equipment and the process for the diagnostic and rehabilitation of a human body locomotor apparatus. More specifically, the present invention relates to an exoskeleton structure to research and exercise the whole upper extremity simultaneously in live multi-joint motion exploring and providing the real data on anatomical torque, forces, and angles in each joint of the extremity.

A human body upper extremity, schematically shown on FIG. 1A, includes four big basic anatomical joints: sternoclavicular, shoulder, elbow and wrist. As shown on FIG. 1A a sternoclavicular joint 101 secures a shouldergirdle 102 to a sternum of a human body (a user's body) 100 with two anatomical rotation axes 103 and 104. An upward-downward motion of the shouldergirdle 102 is provided by its rotation about the axis 104, and a forward-backward motion of the shouldergirdle 102 is provided by its rotation about the axis 103. An upper arm 105 is attached to the shouldergirdle 102 by means of a shoulder joint 106 having three anatomical rotation axes 107, 108, and 109. Flexion-extension, abduction-adduction, and rotation motions of the upper arm 105 are provided by rotation of the upper arm 105 about axes 107, 108, and 109 respectively. A forearm 110 is attached to the upper arm 105 by an elbow joint 111 having two anatomical rotation axes 112 and 114. Flexion-extension and pronation-supination motions of the forearm 110 are provided by rotation of the forearm 110 about axes 112 and 114 respectively. A hand 115 is attached to the forearm 110 by a wrist joint 116 having two anatomical rotation axes 117 and 118. Flexion-extension and abduction-adduction motions of the hand 115 are provided by rotation of the hand 115 about axes 117 and 118 respectively.

The motion stereotype of an individual is formulated by aggregating all the above mentioned motions. The motion stereotype is a complex procedure of muscle interactions that is characterized by minimal expenditure of a muscle energy and defined by a certain launching sequence of different muscles, and dynamic and kinematics parameters of each joint participating in the motion. In medical science, the motion stereotype is an evidence of the normal functioning of the whole organism in general and the upper extremity in particular. Infringement of the motion stereotype is taking place as a result of the muscle contraction change that is the signal of either a larvate or even a developed pathology (in neurology, for example). In a process of the after-stroke physical rehabilitation, for instance, a patient needs to reproduce a precisely coordinated upper extremity motion under control of the biofeedback signals. For assigning a rehabilitation program to a patient after heart attack, local arm isometric muscular work results are used along with the ECG parameters. For defining functional conditions of the patient, ECG, EEG and EMG methods are usually applied in combination with a biomechanical data of the upper extremity functioning.

The most important issue in a diagnosis and a therapy is the exact translation of patient's motion into an exercise device. The better the translation process and the chosen exercise pattern, the more correct the diagnostic results and the more effective physical therapy. And as a result, recovery of the patient will take place faster. It is clear that the trustworthiness of testing and exercising results depends on the exact amount of muscle simultaneous work of the upper extremity segments in every anatomical motion direction.

Methods and equipment for the upper extremity testing and exercising are known. U.S. Pat. No 6,162,190, for example, discloses a method of testing the shouldergirdle-upperarm—forearm kinematically constrained multi-articulated structure with plurality of links, joints and angle position sensors. Axes of the rotation of joints in the structure are coincided with the corresponding anatomical axes of rotation. While the method can be applied for different kinematic tests, it does not include a comprehensive means for exercising and dynamic testing and cannot be used for the diagnosis/rehabilitation programs. An external multi-sectional wireframe attaching to user's body segments for translation of biomechanical parameters into gages and provision an exercise load is usually called an exoskeleton. U.S. Pat. No 6,155,993 discloses an exoskeleton structure comprises a linkage for rotation of two joints in an upper extremity, loading means, angular position sensors, and a harness means attaching the linkage to distal segments of the upper extremity. Linkage axes of articulation in this teaching are parallel, and, thus, the device can be employed only for mono-planar motion of two joints. As well, loading means can not be used independently: a loading dose on one segment depends on the loading dose on another segment. U.S. Pat. No 5,755,645 discloses an exercise apparatus including a multi-link arm with six degree of freedom motion, resistance mechanisms, force sensors, and a harness means. The structure is capable of exercising the complicated functional motions such as throwing a ball or swinging a baseball, but spatial position of joints of the upper extremity cannot be defined, amount of an exercise load is exerted to just one point, and the exercise load cannot be adjusted selectively for each joint of the user's extremity.

The main object of the present invention is to develop a structure and a method that can provide testing and exercising of the whole upper extremity in a realistic manner without an infringement of a user's body locomotor structure, but with a selective biomechanical information and exercise loading for each anatomical motion direction in every joint simultaneously.

Another object of the present invention is to develop a structure to test and exercise both a complex locomotor act and a simple mono-planar motion for both isometric and isotonic muscular contractions.

Another object of the present invention is to develop a structure for determining dynamometry and goniometry parameters selectively in each anatomical motion direction for every joint of the user's upper extremity during a predetermined locomotor act.

Another object of the present invention is to develop a structure to adjust the resistance amount of test/exercise selectively for each anatomical motion direction in every joint of the user's upper extremity.

Another object of the present invention is to develop a modality means having the possibility of reconfiguration of the exoskeleton structure in accordance with predetermined locomotor act, number of links participating in that act, and user's anatomical link size.

### BRIEF SUMMARY OF THE INVENTION

The present invention relates to an exoskeleton structure and a method for testing and exercising of the user's body upper extremity with selectively dosed load and biomechanical measurement of each segment in the upper extremity.

The exoskeleton structure comprises four functional modules: a sternoclavicular, a shoulder, an elbow, and a wrist. Those modules include measuring-loading blocks that are identical, links, and revolute joints. Every measuring-loading block comprises a resistance device to adjust a load for the predetermined exercise motion, a dynamometric device to determine the muscle force, and a goniometric device to measure the joint angle. The shoulder module is connected to the sternoclavicular module, the elbow module is connected to the shoulder module, and the wrist module is connected to the elbow module. In operation the sternoclavicular module is secured to a stationary object. The exoskeleton structure also includes means for connecting modules and for adjustment the distances between joints in accordance with a user's upper extremity link size.

The method for the upper extremity test and exercise comprising a disposition of the exoskeleton structure on the upper extremity wherein all axes of revolute joints in the exoskeleton structure coincide with corresponding axes of anatomical joints of the upper extremity, an adjustment of an exercise resistance for a predetermined exercise motion, and a measurement of a force and an angular displacement of each of the above segments of the upper extremity.

### BRIEF DESCRIPTION OF THE SEVERAL VIEWS OF THE DRAWINGS

The following detailed description of the present invention will be better understood with reference to the accompanying drawings, wherein:

FIG. 1A is a biomechanical schematic view of the upper extremity of a human body;

FIG. 1 is a pictorial view of the exoskeleton structure in accordance with the present invention that is superimposed on the upper extremity of a user sitting on a bench (shown by dotted lines);

FIG. 2 is a pictorial view of the jointed modules of the exoskeleton structure to FIG. 1 with superimposed anatomical axes shown on FIGS. 1A and 1;

FIG. 3 is a pictorial view of a measuring-loading block of the exoskeleton structure to FIG. 2.

FIG. 4 is a cross section view along line A-A on FIG. 3;

FIG. 5 is a cross section view along line B-B on FIG. 3;

FIG. 6 is a pictorial view of the sternoclavicular module of the exoskeleton structure to FIG. 2;

FIG. 7 is a pictorial view of the shoulder module of the exoskeleton structure to FIG. 2;

FIG. 8 is a cross section view of the shoulder module along line C-C on FIG. 7;

FIG. 9 is a cross section view of the shoulder module along line D-D on FIG. 8;

FIG. 10 is a pictorial view of the elbow module of the exoskeleton structure to FIG. 2;

FIG. 11 is a cross section view of the elbow module along line E-E on FIG. 10;

FIG. 12 is a cross section view of the elbow module along line F-F on FIG. 11;

FIG. 13 is a pictorial view of the wrist module of the exoskeleton structure to FIG. 2;

FIG. 14 is an exploded pictorial view of the sternoclavicular coupling to FIG. 2;

FIG. 15 is an exploded pictorial view of the shoulder coupling to FIG. 2;

FIG. 16 is an exploded pictorial view of the elbow coupling to FIG. 2;

FIG. 17 is an exploded pictorial view of the wrist coupling to FIG. 2;

FIG. 18 is a pictorial view of another embodiment of the exoskeleton structure in accordance with the present invention;

FIG. 19 is a pictorial view of another embodiment of the measuring-loading block in the exoskeleton structure to FIG. 18;

FIG. 20 is a cross section view of the measuring-loading block along line G-G to FIG. 19.

### DETAILED DESCRIPTION OF THE INVENTION

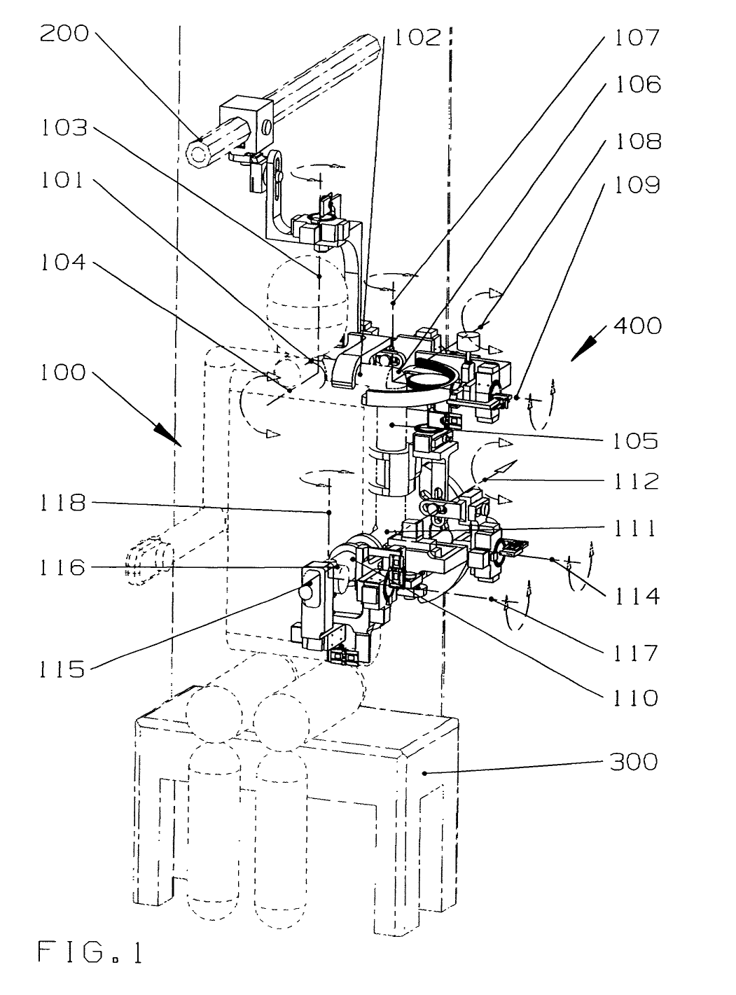

Referring to FIG. 1, an exoskeleton structure (ES) 400 is secured to a stationary object, for example, to a wall mounted polygon beam 200, and attached to the left upper extremity of the user's body 100 located in sitting position on a bench 300. The ES 400 can also be attached to the right upper extremity if required.

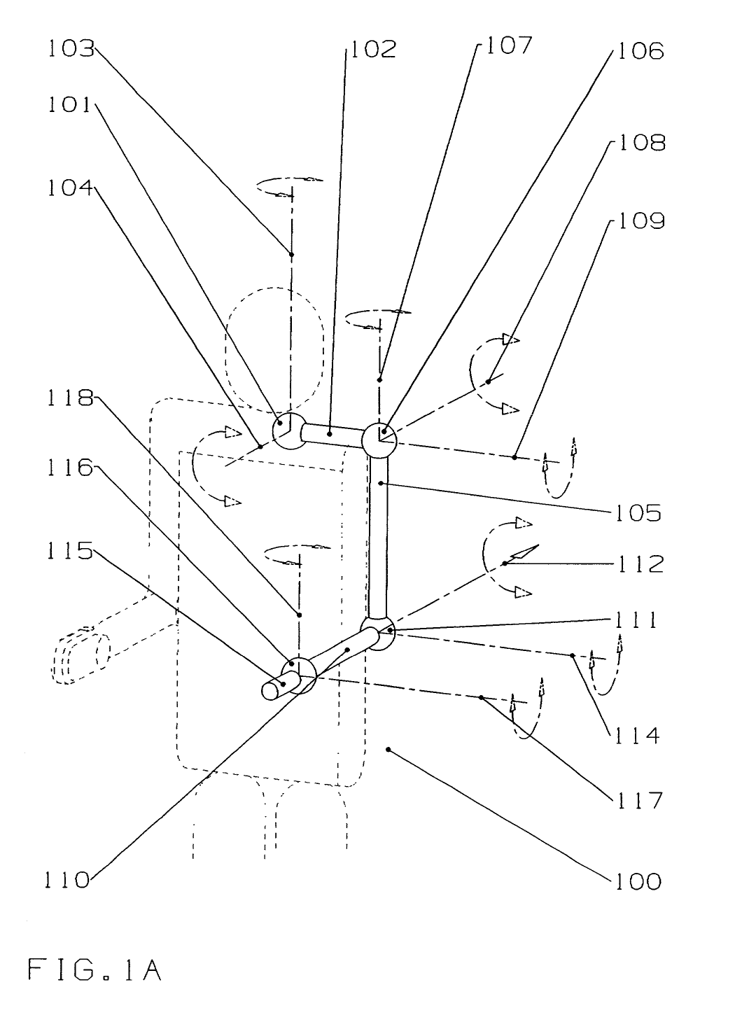

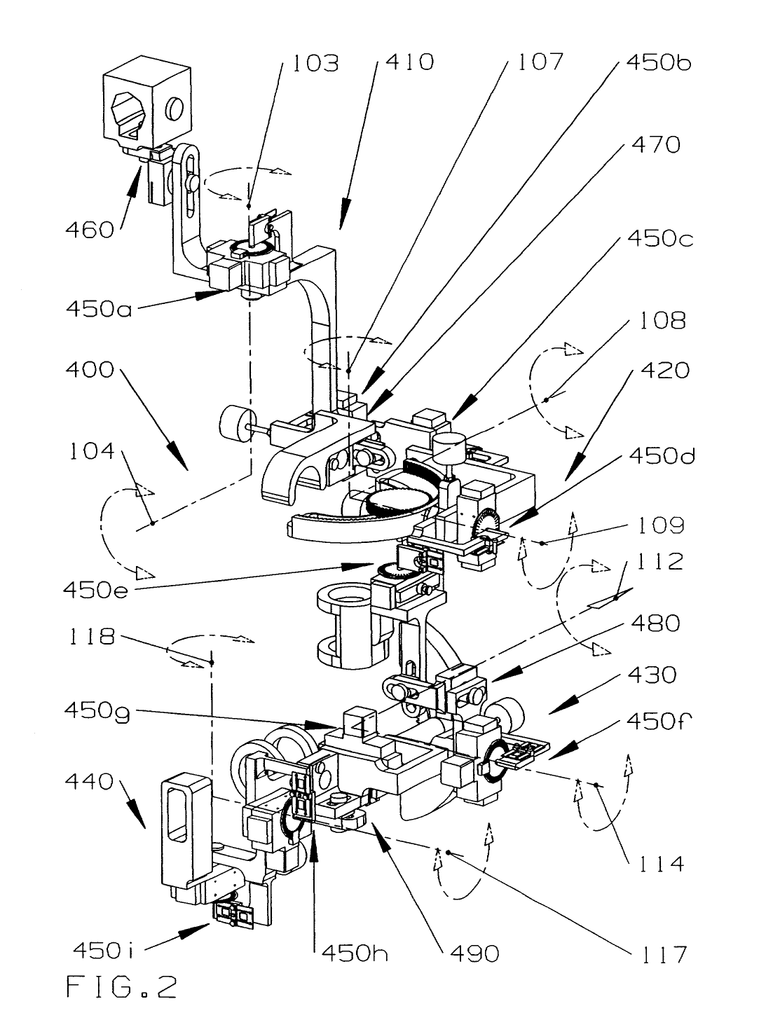

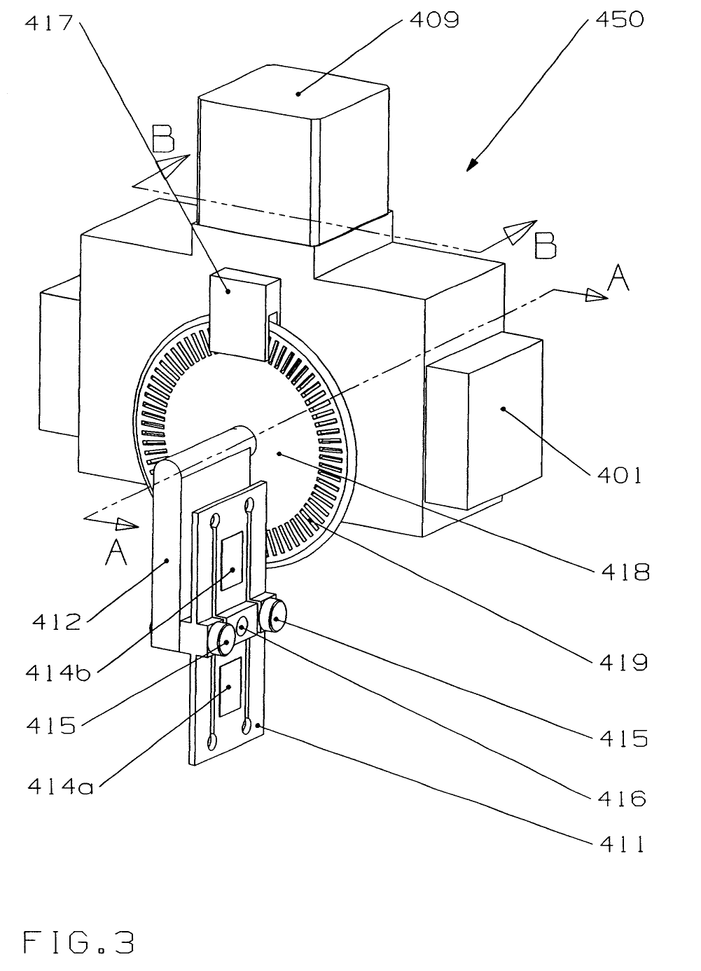

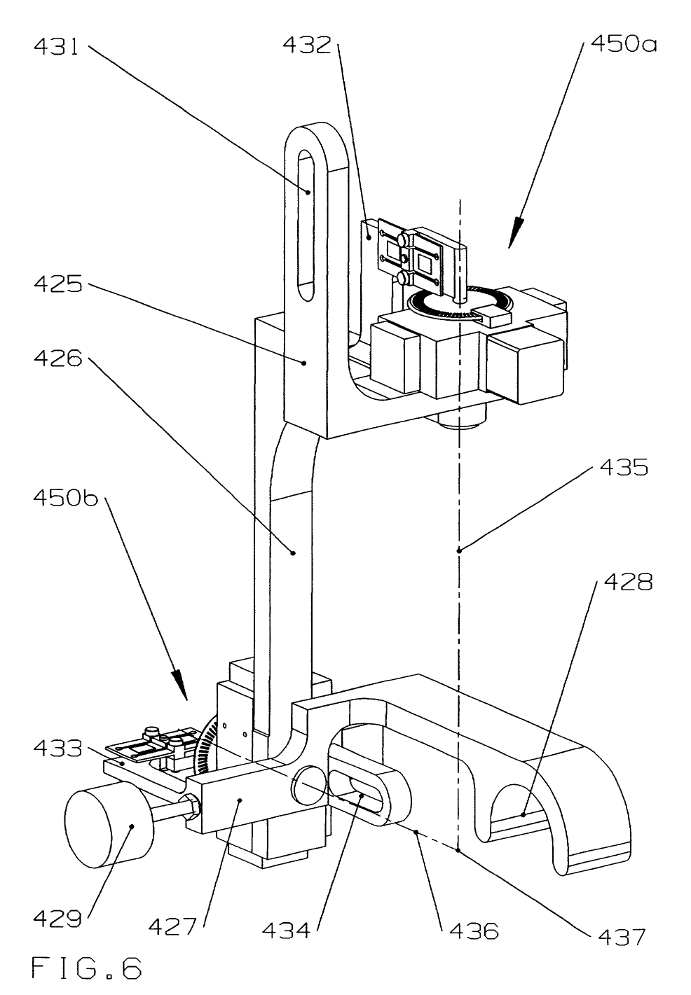

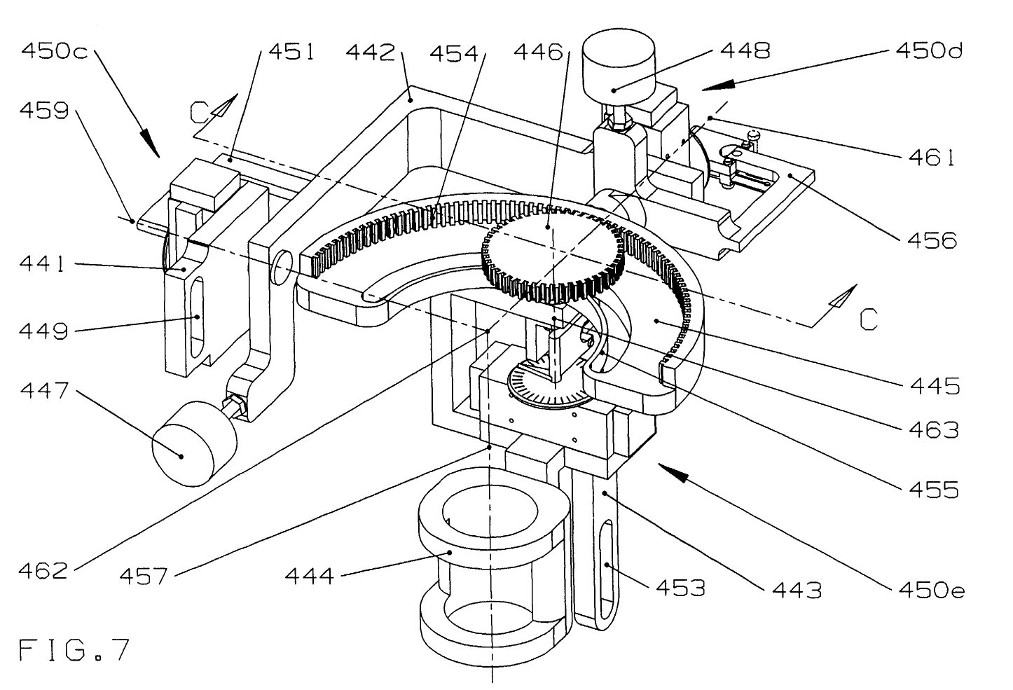

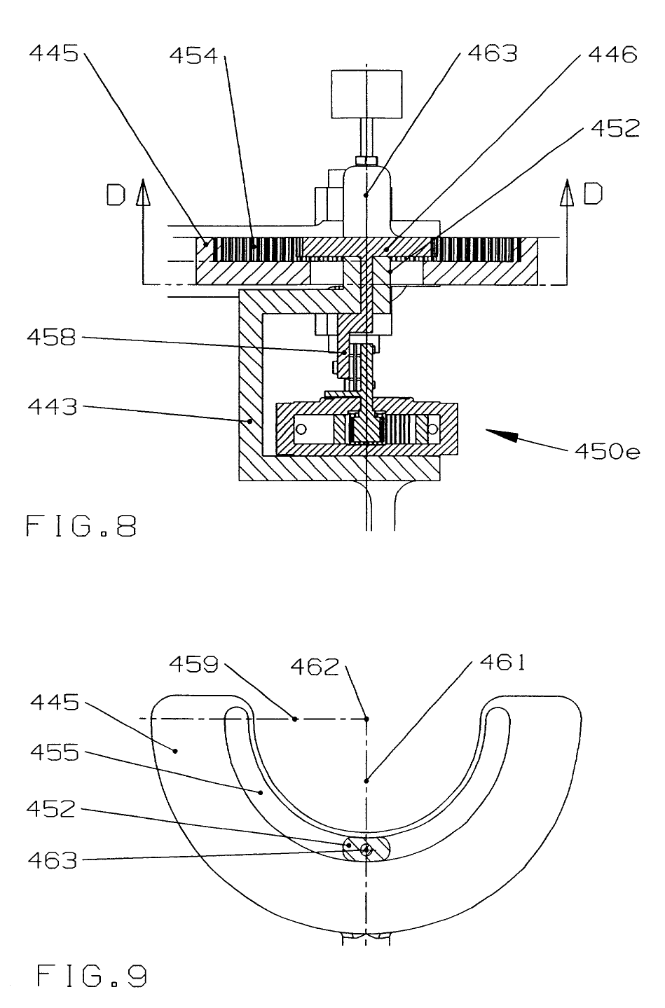

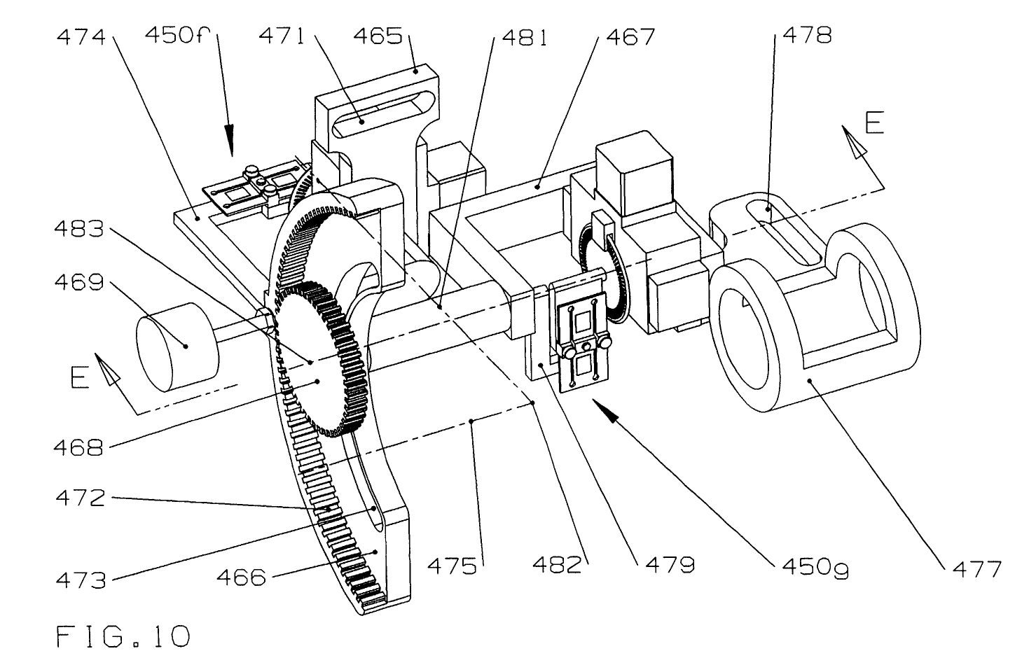

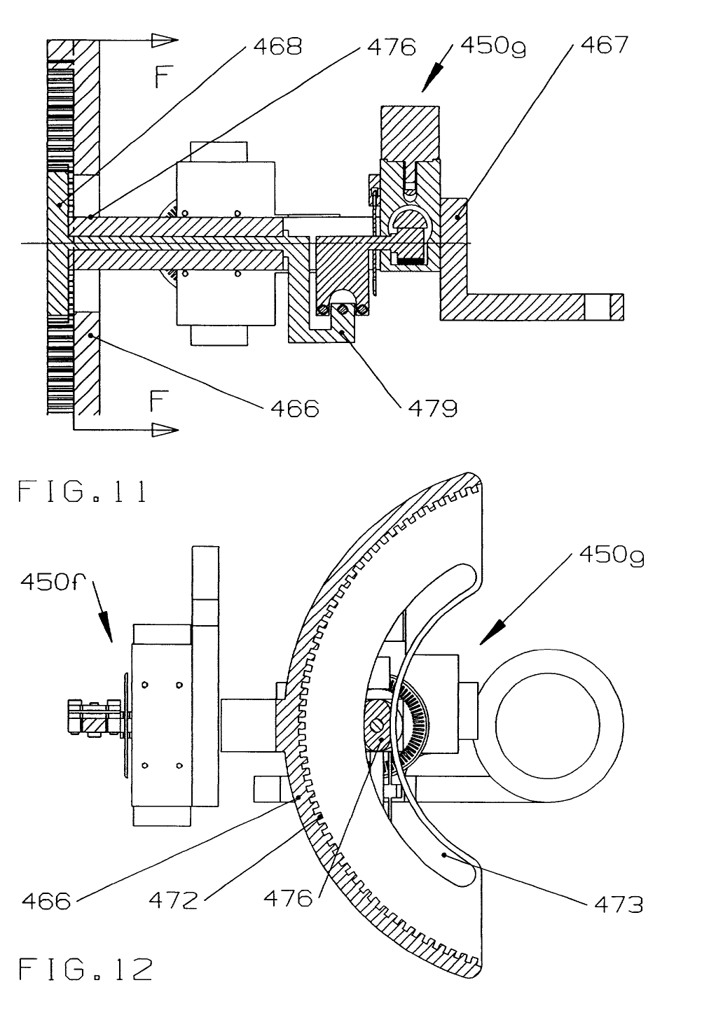

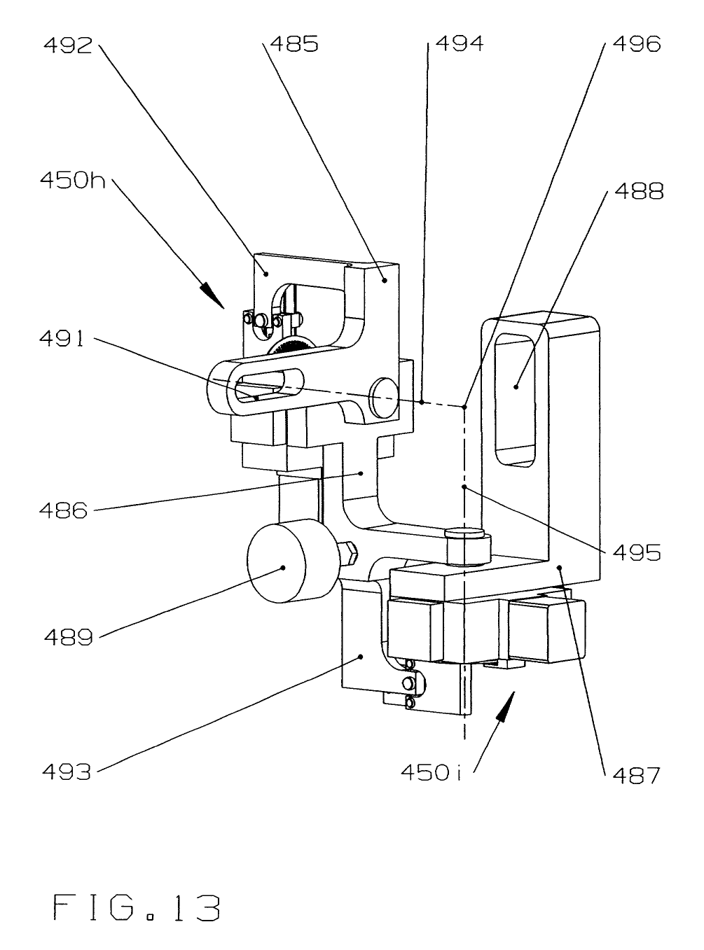

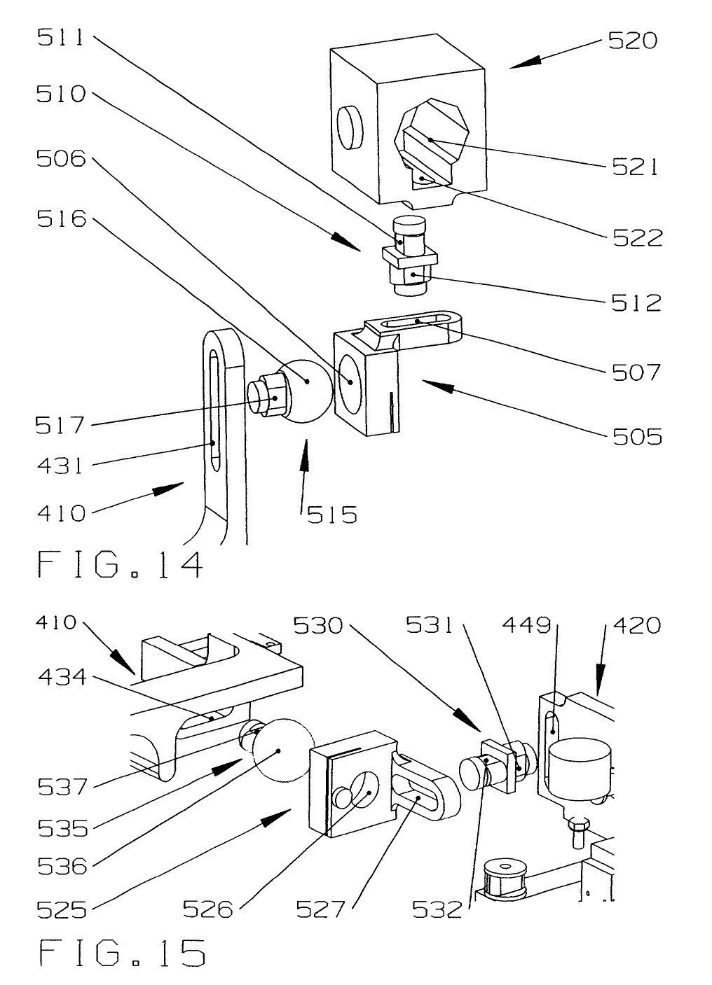

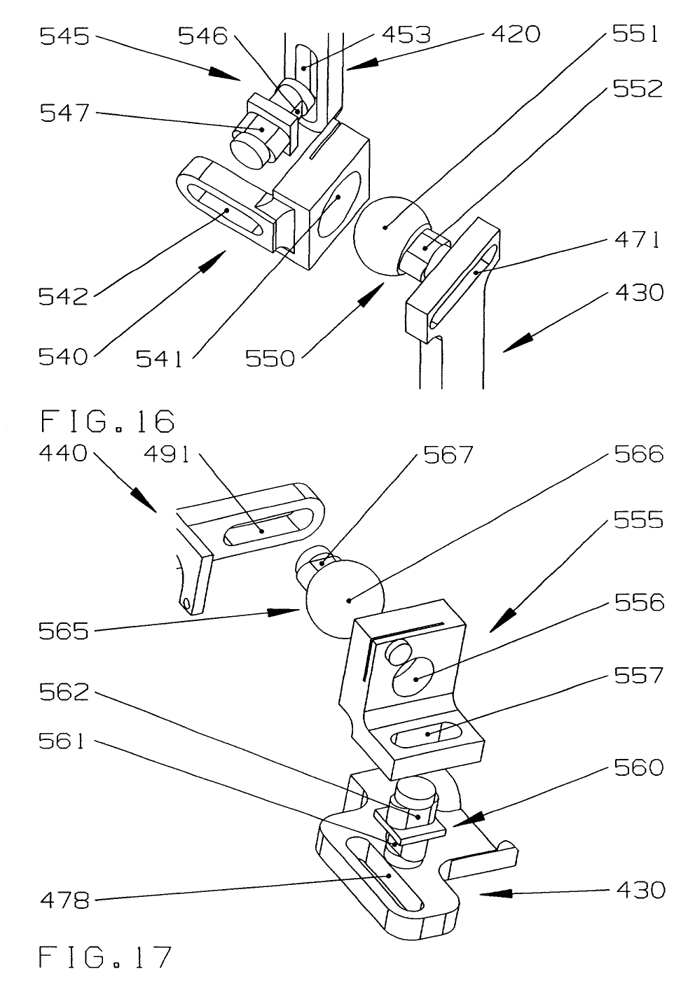

Referring to FIG. 2, the ES 400 comprises a sternoclavicular module 410 (see also FIG. 6), a shoulder module 420 (see also FIGS. 7, 8 and 9), an elbow module 430 (see also FIGS. 10, 11, and 12), and a wrist module 440 (see also FIG. 13). The sternoclavicular module 410 is a two-degree of freedom mechanism having two measuring-loading blocks 450a and 450b (see also FIGS. 3, 4, and 5). The shoulder module 420 is a three-degree of freedom mechanism having three measuring-loading blocks 450c, 450d and 450e. The elbow module 430 is a two-degree of freedom mechanism having two measuring-loading blocks 450f and 450g. The wrist module 440 is a two-degree of freedom mechanism having two measuring-loading blocks 450h and 450i. In operation, the sternoclavicular module 410 should be secured to a stationary object by means of a sternoclavicular coupling 460, for example, to the polygon wall-mounted beam 200 (see also FIG. 14). The shoulder module 420 is connected to the sternoclavicular module 410 by a shoulder coupling 470 (see also FIG. 15). The elbow module 430 is connected to the shoulder module 420 by an elbow coupling 480 (see also FIG. 16). The wrist module 440 is connected to the elbow module 430 by a wrist coupling 490 (see also FIG. 17).

All measuring-loading blocks (MLB) 450a-450i are identical. They are shown on FIGS. 3, 4, and 5 generally as 450, and enlarged for clarity. Every MLB comprises a resistance device to adjust the testing/exercising load, a dynamometric device to determine the muscle force, and a goniometric device to measure the joint angle. The resistance device includes a housing 401 having hydraulic chambers 402 and 403 connected by channel 404, a piston 405 with a gear rack 406, and a gear shaft 407. The gear shaft 407 is engaged with the gear rack 406 and installed in the housing 401 wherein it can be rotated. The piston 405 can be moved inside the housing 401 in such a way that chambers 402 and 403 always stay hydraulically connected with a channel 404. The chambers 402 and 403 and the channel 404 are filled up with the incompressible fluid. The cross-section area of the channel 404 can be adjusted by a valve 408 that can be activated by a stepper motor 409 electrically connected to a computer system (not shown). The smaller the cross-section area of the channel 404 the higher resistance to move the piston 405. The dynamometric device includes an elastic element 411 having very small elastic deformation, and support 412 secured to the gear shaft 407. The dynamometric device also includes four strain gages 414a-414d that are affixed to the elastic element 411. Gages 414a-414d are electrically connected in a way to form a Wheatstone bridge circuit (not shown) providing an electrical signal proportional to the elastic element 411 deformation. Distal center parts of the elastic element 411 are mounted to the support 412 by screws 415 An inner center part of the elastic element 411 has a hole 416 for connection with the modules 410, 420, 430, and 440. The goniometric device involves an index indicator 417 (for example a LEO type) secured to the housing 411 and electrically connected to the computer system (not shown). This device also includes a disk 418 with sircumferentially located slits 419. The disk 419 is secured to the gear shaft 407. The gear shaft 407, the support 412, and the disk 418 have a common rotation axis 421. In operation, the testing/exercising load applies to the inner central part of the elastic element 411. The elastic element 411 together with the elastic element support 412, the gear shaft 407, and the disk 418 can be rotated about the axis 421 overcoming the resistance force against a motion of the piston 405 which is defined by the valve 408. The computer system reads data from the strain gages 414a-414d and the indicator 417.

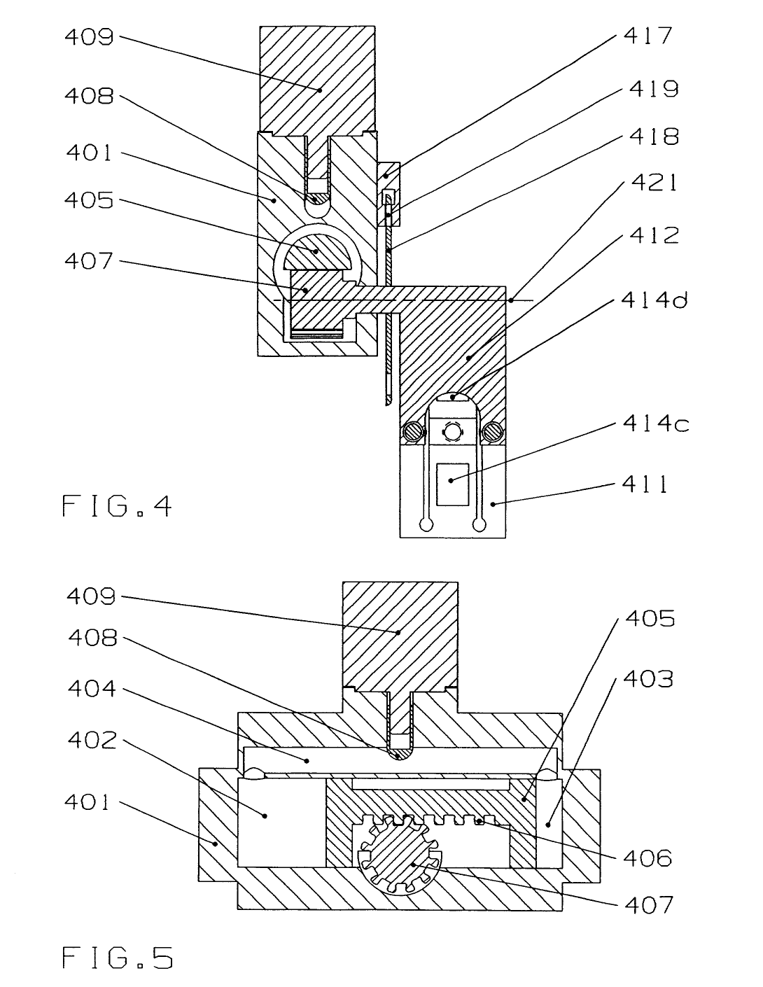

Referring to FIG. 6, the sternoclavicular module 410 consists of two MLBs 450a and 450b, a first sternoclavicular brace 425, a sternoclavicular bracket 426, a second sternoclavicular brace 427 with a shouldergirdle harness 428, and a sternoclavicular counter weight 429 installed on the second sternoclavicular brace 427 to balance the force of gravitation. The first sternoclavicular brace 425 has a first sternoclavicular slot 431 to secure the sternoclavicular module 410 to a stationary object, for example, the wall-mounted beam 200 by means of the sternoclavicular coupling 460. The sternoclavicular bracket 426 has a first sternoclavicular extension 432. The second sternoclavicular brace 427 has a second sternoclavicular extension 433 and a second sternoclavicular slot 434 to secure the sternoclavicular module 410 to the shoulder module 420 by means of the shoulder coupling 470. The first sternoclavicular brace 425 and the second sternoclavicular brace 427 are mounted on the sternoclavicular bracket 426 and can be rotated around a first sternoclavicular axis 435 and a second sternoclavicular axis 436 respectively. The first sternoclavicular axis 435 intersects the second sternoclavicular axis 436 at the 90-degree angle in a sternoclavicular point 437. The housing 401 of the MLB 450a is secured to the first sternoclavicular brace 425, such that the axis 421 (see FIG. 4) is coaxial with the first sternoclavicular axis 435. The elastic element 411 is attached to the first sternoclavicular extension 431 through the hole 416. The housing 401 of the MLB 450b is fixed to the sternoclavicular bracket 426 such that the axis 421 is coaxial with the second sternoclavicular axis 436. The elastic element 411 is attached to the second sternoclavicular extension 433.

Referring to FIGS. 7, 8, and 9, the shoulder module 420 consists of three MLBs 450c, 450d and 450e, a first shoulder brace 441, a shoulder bracket 442, a second shoulder brace 443 with an upper arm harness 444, a shoulder arch bracket 445, a shoulder planet gear 446, a first shoulder counter weight 447 installed on the shoulder bracket 442, and a second shoulder counter weight 448 installed on the shoulder arch bracket 445. The first shoulder brace 441 comprises a first shoulder slot 449 to secure the shoulder module 420 to the sternoclavicular module 410 by means of the shoulder coupling 470. The shoulder bracket 442 has a first shoulder extension 451. The second shoulder brace 443 has a shoulder slides 452 and a second shoulder slot 453 to secure the shoulder module 420 to the elbow module 430 by means of the elbow coupling 480. The shoulder arch bracket 445 includes a shoulder internal-toothed gear 454, a shoulder arch slot 455, and a second shoulder extension 456. The shoulder internal-toothed gear 454, and the shoulder arch slot 455 have mutual a first shoulder axis 457. The shoulder planet gear 446 has a third shoulder extension 458. The first shoulder brace 441 and the shoulder arch bracket 445 are mounted on the shoulder bracket 442 and can be rotated around a second shoulder axis 459 and a third shoulder axis 461 respectively. The second shoulder axis intersects the third shoulder axis 461 at 90 angle in a shoulder point 462. The shoulder slide 452 is installed in the shoulder arch slot 455 and can be moved along the shoulder arch slot 455. The shoulder planet gear 446 is installed in the shoulder slides 452, can be rotated into the shoulder slides 452 around a shoulder auxiliary axis 463, can be moved along the shoulder arch slot 455 together with the shoulder slide 452, and permanently engaged with the shoulder internal-toothed gear 454. The first shoulder axis 457 intersects the second shoulder axis 459 and the third shoulder axis 461 at 90 degree angle in the shoulder point 462. The housing 401 of the MLB 450c is secured to the first shoulder brace 441 such that the axis 421 is coaxial with the first shoulder axis 459. The elastic element 411 is attached to the first shoulder extension 451 through the hole 416. The housing 401 of the MLB 450d is secured to the shoulder bracket 442 such that the axis 421 is coaxial with the third shoulder axis 461. The elastic element 411 is attached to the second shoulder extension 456 through the hole 416. The housing 401 of the MLB 450e is secured to the second shoulder brace 443 such that the axis 421 is coaxial with the shoulder auxiliary axis 463. The elastic element 411 is attached to the third shoulder extension 458 through the hole 416.

Referring to FIGS. 10, 11, and 12, the elbow module 430 consists of two MLBs 450f and 450g, a first elbow brace 465, an elbow arch bracket 466, a second elbow brace 467, an elbow planet gear 468, and an elbow counter weight 469 installed on the elbow arch bracket 466. The first elbow brace 465 has a first elbow slot 471 to secure the elbow module 430 to the elbow coupling 480. The elbow arch bracket 466 comprises an elbow internal-toothed gear 472, an elbow arch slot 473, and a first elbow extension 474. The elbow internal-toothed gear 472 and the elbow arch slot 473 have mutual a first elbow axis 475. The second elbow brace 467 has an elbow slides 476, a forearm harness 477, and a second elbow slot 478 to secure the elbow module 430 to the wrist coupling 490. The elbow planet gear 468 includes a second elbow extension 479. The first elbow brace 465 is mounted on the elbow arch bracket 466 and can be rotated around a second elbow axis 481. The second elbow axis 481 intersects the first elbow axis 475 at 90-degree angle in an elbow point 482. The elbow slide 476 is installed in the elbow arch slot 473 and can be moved along the elbow arch slot 473. The elbow planet gear 468 is installed in the elbow slides 476, can be rotated into the elbow slides 476 around an elbow auxiliary axis 483, can be moved along the elbow arch slot 473 together with the elbow slide 476, and permanently engaged with the elbow internal-toothed gear 472. The housing 401 of the MLB 450f is secured to the first elbow brace 465 such that the axis 421 is coaxial with the second elbow axis 481. The elastic element 411 is attached to the first elbow extension 474 through the hole 416. The housing 401 of the MLB 450g is secured to the second elbow brace 467 such that the axis 421 is coaxial with the elbow auxiliary axis 483. The elastic element 411 is attached to the second elbow extension 479 through the hole 416.

Referring to FIG. 13, the wrist module 440 consists of two MLBs 450h and 450i, a first wrist brace 485, a wrist bracket 486, a second wrist brace 487 with a hand harness 488, and a wrist counter weight 489 installed on the wrist bracket 486 to balance the force of gravitation. The first wrist brace 485 has a wrist slot 491 to secure the wrist module 440 to the elbow module 430 by means of the wrist coupling 490. The first wrist brace 485 has a first wrist extension 492. The wrist bracket 486 has a second wrist extension 493. The first wrist brace 485 and the second wrist brace 487 are mounted on the wrist bracket 486 and can be rotated around a first wrist axis 494 and a second wrist axis 495 respectively. The first wrist axis 494 intersects a second wrist axis 495 at the 90-degree angle in the wrist point 496. The housing 401 of the MLB 450h is secured to the wrist bracket 486 such that the axis 421 is coaxial with the first wrist axis 494. The elastic element 411 is attached to the first wrist extension 492 through the hole 416. The housing 401 of the MLB 450i is fixed to the second wrist brace 487 such that the axis 421 is coaxial with the second wrist axis 495. The elastic element 411 is attached to the second wrist extension 493 through the hole 416.

Referring to FIG. 14, the sternoclavicular coupling 460 consists of a sternoclavicular coupling brace 505, a first sternoclavicular coupling adapter 510, a second sternoclavicular coupling adapter 515 and a sternoclavicular coupling mount 520. The sternoclavicular coupling brace 505 comprises a sternoclavicular spherical hole 506 and a sternoclavicular coupling slot 507. The first sternoclavicular coupling adapter 510 includes a first sternoclavicular slide 511 and a second sternoclavicular slide 512. The second sternoclavicular coupling adapter 515 has an outer sternoclavicular sphere 516 and a third sternoclavicular slide 517. The sternoclavicular coupling mount 520 has a rotation preventing orifice 521, for example, of a polygonal shape, and a mount coupling slot 522. The first sternoclavicular coupling adapter 510 is secured to the sternoclavicular coupling brace 505 by means of the second sternoclavicular slide 512 that can be moved along the sternoclavicular coupling slot 507. The second sternoclavicular coupling adapter 515 is secured to the sternoclavicular coupling brace 505 by means of the outer sternoclavicular sphere 516 that can be rotated in the sternoclavicular spherical hole 506. The second sternoclavicular coupling adapter 515 is secured to the first slot 431 of the sternoclavicular module 410 by means of the third sternoclavicular slide 517 which can be moved along the slot 431. The first sternoclavicular coupling adapter 510 is secured to the sternoclavicular coupling mount 520 by means of the first sternoclavicular slide 511, which can be moved along the mount coupling slot 522. The sternoclavicular coupling mount 520 is secured to a stationary object, for example, to a polygonal wall-mounted beam 200 by means of the polygonal orifice 521.

Referring to FIG. 15, the shoulder coupling 470 consists of a shoulder coupling brace 525, a first shoulder coupling adapter 530, and a second shoulder coupling adapter 535. The shoulder coupling brace 525 comprises a shoulder spherical hole 526 and a shoulder coupling slot 527. The first shoulder coupling adapter 530 includes a first shoulder coupling slide 531 and a second shoulder coupling slide 532. The second shoulder coupling adapter 535 has an outer shoulder sphere 536 and third shoulder coupling slide 537. The first shoulder coupling adapter 530 is secured to the shoulder coupling brace 525 by means the second shoulder coupling slide 532 which can be moved along the shoulder coupling slot 527. The second shoulder coupling adapter 535 is secured to the shoulder coupling brace 525 by means of the outer shoulder sphere 536 which can be rotated in the shoulder spherical hole 526. The second shoulder coupling adapter 535 is secured to the second sternoclavicular slot 434 of the sternoclavicular module 410 by means of the third shoulder coupling slide 537 which can be moved along the slot 434. The shoulder module 2 is secured to the first shoulder coupling adapter 530 by means of the first shoulder coupling slide 531 which can be moved along the first slot 449 of the shoulder module 420.

Referring also to FIG. 16, the elbow coupling 480 consists of an elbow coupling brace 540, a first elbow coupling adapter 545, and a second elbow coupling adapter 550. The elbow coupling brace 540 comprises an elbow spherical hole 541 and a brace elbow coupling slot 542. The first elbow coupling adapter 545 includes a first elbow coupling slide 546 and a second elbow coupling slide 547. The second elbow coupling adapter 550 has an outer elbow sphere 551 and a third elbow coupling slide 552. The first elbow coupling adapter 545 is secured to the elbow coupling brace 540 by means of the second elbow coupling slide 547 which can be moved along the brace elbow coupling slot 542. The second elbow coupling adapter 550 is secured to the elbow coupling brace 540 by mean of the outer elbow sphere 551 which can be rotated into the elbow spherical hole 541. The first elbow coupling adapter 545 is secured to the second shoulder slot 453 of the shoulder module 420 by mean of the first elbow coupling slide 546 which can be moved along the slot 453. The elbow module 430 is secured to the second elbow coupling adapter 550 by means of the third elbow coupling slide 552 which can be moved along the first elbow slot 471 of the elbow module 430.

Referring also to FIG. 17, the wrist coupling 490 consists of a wrist coupling brace 555, a first wrist coupling adapter 560, and a second wrist coupling adapter 565. The wrist coupling brace 555 comprises a wrist spherical hole 556 and a brace wrist coupling slot 557. The first wrist coupling adapter 560 includes a first wrist coupling slide 561 and a second wrist coupling slide 562. The second wrist coupling adapter 565 has an outer wrist sphere 566 and a third wrist coupling slide 567. The first wrist coupling adapter 560 is secured to the wrist coupling brace 555 by means of the second wrist coupling slide 562 which can be moved along the brace wrist coupling slot 557. The second wrist coupling adapter 565 is secured to the wrist coupling brace 555 by means of the outer wrist sphere 566 which can be rotated into the wrist spherical hole 556. The first wrist coupling adapter 560 is secured to the second elbow slot 478 of the elbow module 430 by means of the first wrist coupling slide 561 which can be moved along the slot 478. The wrist module 440 is secured to the second wrist coupling adapter 565 by means of the third wrist coupling slide 567 which can be moved along the wrist slot 491 of the wrist module 440.

In operation, before testing/exercising, the ES 400 is disposed on the user 100 in such a way, that the first sternoclavicular axis 435 and the second sternoclavicular axis 436 of the sternoclavicular module 410 are placed to coincide with anatomical rotation axes 103 and 104 of the sternoclavicular joint 101 respectively (see FIGS. 1 and 6). Therefore, the sternoclavicular point 437 coincides with the anatomical center of rotation in the sternoclavicular joint 101. To provide this setting of the ES 400 the mount 520 (see FIG. 14) is secured to a stationary object, for example, to a wall mounted polygonal beam 200. The first adapter 510 together with the brace 505, the second adapter 515, and the sternoclavicular module 410 are moved relative to the mount 520 in the slot 522 of the mount 520. The brace 505 together with the second adapter 515 and the sternoclavicular module 410 are moved relative to the first adapter 510 in the second slide 512 of the first adapter 510. The second adapter 515 together with the sternoclavicular module 410 is rotated relative to the brace 505 in the spherical hole 506 of the brace 505. The sternoclavicular module 410 is moved relative to the second adapter 515 in the third slide 517 of the second adapter 515. Those transpositions can be either apart or simultaneously. After that the required position of the sternoclavicular module 410 is affixed (fixing elements of the sternoclavicular coupling 460, the shoulder coupling 470, the elbow coupling 480, and the wrist coupling 490 are not shown for clarity). Then, the shouldergirdle harness 428 is secured to a shouldergirdle 102 by a belt (not shown).

The shoulder module 420 is installed in such a way that first, second, and third shoulder axes 457, 459, and 461 of the shoulder module 420 are placed to coincide with the anatomical rotation axes 107, 109, and 108 of the shoulder joint 106 respectively (see FIGS. 1 and 7). Therefore, the shoulder point 462 coincides with the anatomical center of rotation in the shoulder joint 106. In order to accomplish those coincidences, like so as for the sternoclavicular module 410, the brace 525 (see FIG. 15) together with the shoulder module 420 are rotated and moved relative to the sternoclavicular module 410 by means of the adapter 536, and the shoulder module 420 is moved relative to the brace 525 by means of the adapter 530. Thus, the required position of the shoulder module 420 of the ES 400 on the user's upper extremity is achieved and affixed. Then, the shoulder harness 444 is secured to the upper arm 105 by a belt (not shown).

The elbow module 3 is installed in such a way that first and second elbow axes 475 and 481 of the elbow module 430 are placed to coincide with the anatomical rotation axes 112 and 114 of the elbow joint 111 respectively (see FIGS. 1 and 10). Therefore, the elbow point 482 coincides with the anatomical center of rotation of the elbow joint 111. To accomplish this situation, like so as for the sternoclavicular module 410, the brace 540 (see FIG. 16) together with elbow module 430 are moved relative to the shoulder module 420 by means of the adapter 545, and the elbow module 430 is rotated and moved relative to the brace 540 by means of the adapter 550. When the required position of the elbow module 430 is achieved, that position is affixed, and the elbow harness 477 is secured to a forearm 110 by a belt (not shown).

The wrist module 440 is installed in such a way that first and second elbow axes 494 and 495 of the wrist module 440 are placed to coincide with the anatomical rotation axes 117 and 118 of the wrist joint 116 respectively (see FIGS. 1 and 13). Therefore, the wrist point 496 coincides with the anatomical center of rotation in the wrist joint 116. For this, like so as for the sternoclavicular module 410, the brace 555 (see FIG. 17) together with the wrist module 410 are rotated and moved relative to the elbow module 430 by means of the adapter 565, and the wrist module 440 is moved relative to the brace 555 by means of the adapter 560. Thus, the required position of the wrist module 440 of the ES 400 is achieved and affixed. Then, the hand harness 488 is secured to the user's hand 115 by a belt (not shown).

Muscular contractions have two basic regimes: isometric in which the length of the muscle remains constant while the muscle works against resistance and isotonic in which the muscle remains under relatively constant tension while its length changes. Thus, in an isometric mode exercise, positions of the user's segment are not changed, and muscles cannot overcome exercising resistance. In an isotonic mode exercise, positions of the user's segments are changed because exercising resistance is less then muscle forces.

For testing/exercising in the isometric mode, the predetermined space position of module rotation points 437, 462, 482, and 496 and its axes, which determine joint angles of the user's arm, have to be installed and fixed. For providing an easy those operations, in each MLB 450a-450i (see FIGS. 3, 4, and 5) the cross section area of the channel 404 should be wide opened by the valve 408. The support 412 together with the elastic element 411 and the disk 418 are rotated about the axis 421, and the indicator 417 reads the required joint angle. Because the elastic element 411 is connected with corresponding extension in every module, those extensions are turned together with the corresponding parts of the modules 410-440. To affix installed angle, the cross-section area of the channel 404 is filly blocked by the valve 408, and the piston 405 is not able to move.

In a case of a multi-joint complex locomotor act, the user attempts to execute a predetermined arm motion, for example, to take a piece of paper from a table. As mentioned above, the elastic element 411 has a very small elastic deformation. Because of this, live motions of the shouldergirdle 102, the upperarm 105, the forearm 110 and the hand 115 will not occur. But almost all muscles of the user's arm participate in isometric muscular contractions, and all MLBs 450a-450i are affected.

In a case of a mono-joint locomotor act, for instance, the upperarm abduction-adduction, the user attempts to rotate the upperarm 105 about the axis 108 outward and inward relative to his torso. Remainder segments of the arm and muscles are enervated. For the upperarm abduction-adduction the MLB 450c only is affected.

In the isotonic mode of operation, for each MLB 450a-450i a predetermined testing/exercising load is fixed by adjusting the valve 408. In this mode of operation the testing/exercising load in the MLBs 450a-450i can be either the same or vary according to the testing/exercising program.

In the case of a multi-joint complex locomotor act, the user executes a predetermined arm movement, for example, to take and move a piece of paper on a table. Almost all muscles and segments of the arm participate in that live motion. All MLBs 450a-450i are affected in the same way as for the case of the isometric multi-joint complex locomotor act. The channel 404 is not blocked in this mode and the gear shaft 407 with the support 412 and the elastic element 411 can be rotated. When the corresponding extension exerts the elastic element 411, the last is deformed. When the user overcomes the testing/exercising load, the elastic element 411 starts to turn together with the disk 418.

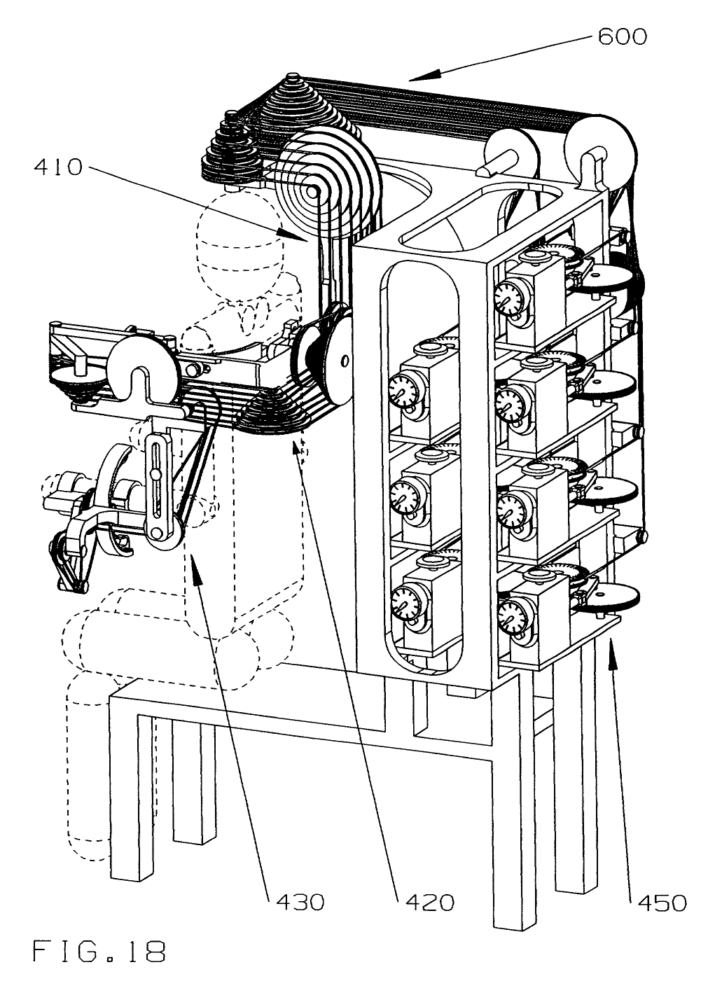

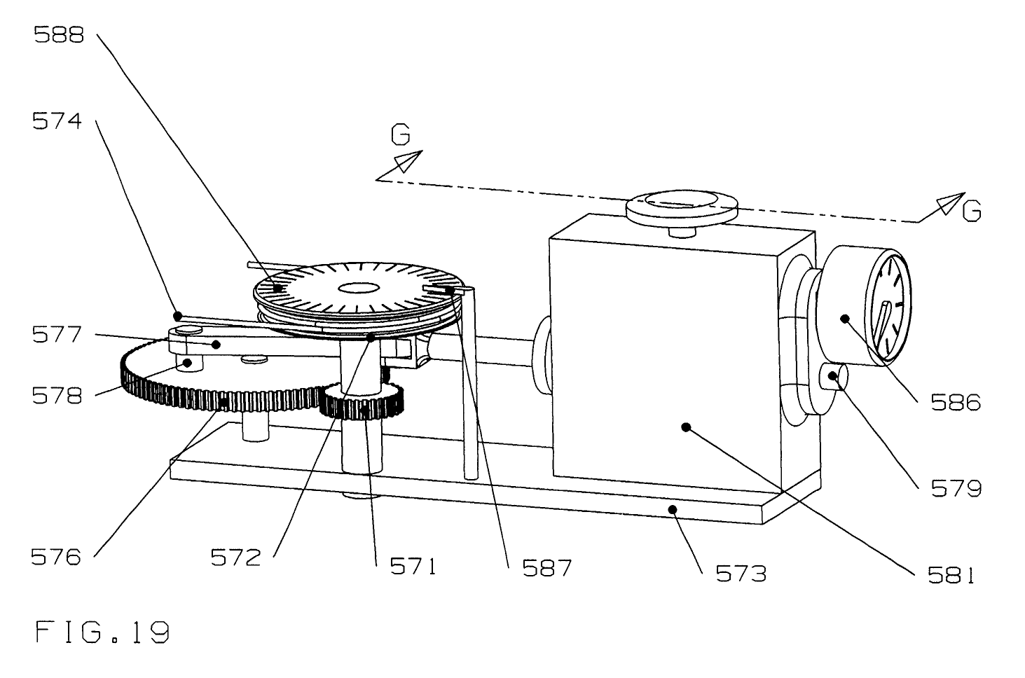

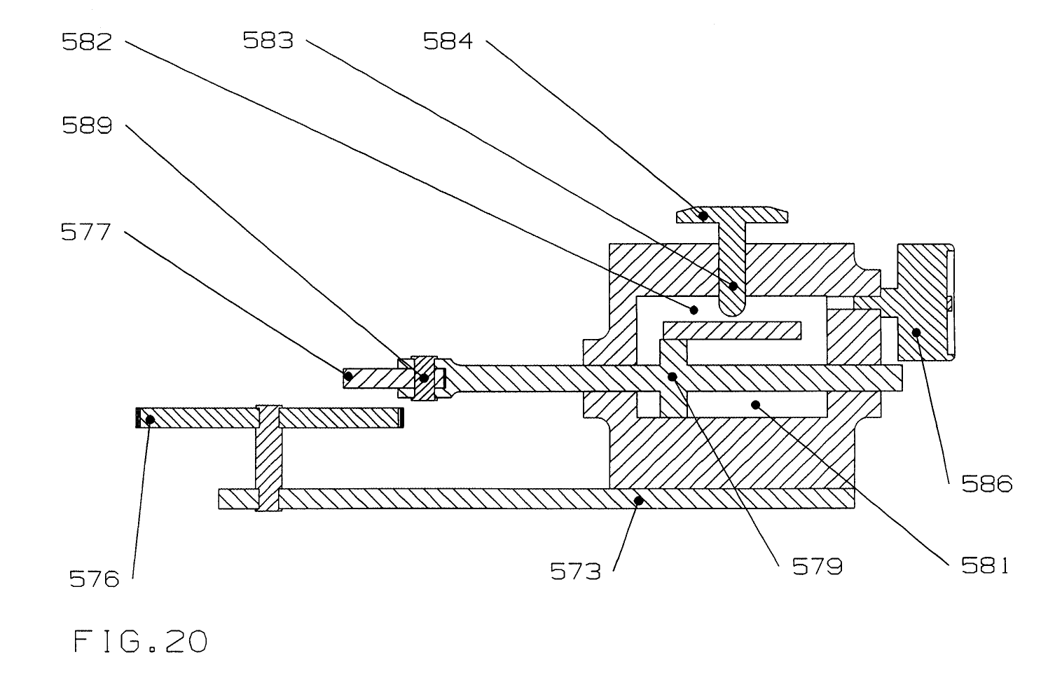

On FIGS. 18-20, the second embodiment of the present invention is shown. In this embodiment each of the rotation axis of the modules 410, 420, and 430 is interconnected with corresponding rotation axis of the MLB 450 by means of a cable system 600. The resistance device (see FIGS. 19 and 20) comprises a driving gear 571 and a pulley 572. The gear 571 and the pulley 572 are immovable relative to each other but can be rotated in a base 573. The pulley 572 can receive a cable 574. The driving gear 571 is permanently engaged with a driven gear 576. The driven gear 576 is jointed to the first end of a connection rod 577 through a pin 578. The second end of the connecting rod 577 is jointed to the piston 579 through a pin 589. The piston 579 can be moved in a cylinder 581 between its front and rear ends. The cylinder 581 is secured to the base 573. The cylinder 581 having a channel 582 is filled in operation with incompressible fluid. The channel 582 connects the front and the rear ends of the cylinder 581, and can not be blocked by the piston 579. The cross-section area of the channel 582 can be adjusted by a valve 583 that is manually operated by a handle 584. To take a muscle force and/or exercise load reading, a pressure gage 586 is installed in the cylinder 581. The goniometric device involves an arrow 587 secured to the base 573 and a scale 588 affixed to the pulley 572 to take a joint angle alteration reading,

Thus, the exoskeleton structure, according to the present invention, is able to provide testing and exercising of whole upper extremity of a user in a realistic manner. The exoskeleton structure can provide a biomechanical information about a user's upper extremity either simultaneously for all the joints or selectively for a joint of interest. The exoskeleton structure also can read the information either about a single anatomical motion of the user's upper extremity or a combination of anatomical motions of any complexity. Based on the information, the apparatus can provide a dosed load to user's given motions, and by this a proper exercise and rehabilitation program can be achieved.

While the exoskeleton structure for upper extremity testing and exercising according to the invention have been described in details above, it is clear that there are variations and modifications to this disclosure here and above which will be readily apparent to one of the ordinary skills in the art. To the extent that such variations and modifications of the present disclosure of the exoskeleton structure, wherein all axes of joint rotation in the exoskeleton structure are coincided with corresponding anatomical joints of a user's upper extremity, and all portions in the exoskeleton structure are interconnected so as described in the present disclosure, such are deemed within the scope of the present invention.

We claim:

1. An upper extremity exoskeleton structure for test and exercise comprising:
a plurality of links, a plurality of revolute joints, and a plurality of measuring-loading blocks wherein each said measuring-loading block having a resistance mechanism, a force sensor, and an angle sensor connected with said links through said revolute joints, and said links jointed to each other by said revolute joints forming a sternoclavicular, a shoulder, an elbow and a wrist modules;
said sternoclavicular module includes a shouldergirdle harness, two said measuring-loading blocks, two said revolute joints and said links wherein rotation axes of said revolute joints intersect each other at 90-degree angle;
said shoulder module includes an upper arm harness, three said measuring-loading blocks, three said revolute joints and said links wherein said rotation axes of said revolute joints intersect each other at 90-degree angle;
said elbow module includes a forearm harness, two said measuring-loading blocks, two said revolute joints and said links wherein said rotation axes of said revolute joints intersect each other at 90-degree angle;
said wrist module includes a hand harness, two said measuring-loading blocks, two said revolute joints and said links wherein said rotation axes of said revolute joints intersect each other at 90-degree angle;
a plurality of means to connect said modules with each other forming said exoskeleton structure;
a plurality of means to compensate anthropometrical differences of the upper extremities between different users;
a means to secure said exoskeleton structure to a stationary object;
a plurality of means to counterbalance said exoskeleton structure.

2. An exoskeleton structure to claim 1,
wherein said rotation axes of said exoskeleton structure, being secured to a user's upper extremity, intersect each other in the center of the user's sternoclavicular joint for said sternoclavicular module, in the center of the user's shoulder joint for said shoulder module, in the center of the user's elbow joint for said elbow module, and in the center of the user's wrist joint for said wrist module.

3. An exoskeleton structure to claim 1,
wherein number of said modules connected by said plurality of connecting means forming said exoskeleton structure is changeable in accordance with the predetermined locomotor act and user's joints participating in that locomotor act.

4. A method for an upper extremity test and exercise comprising:
a disposition of said exoskeleton structure on said user's upper extremity in such a way where each said rotation axis of said modules coincides with corresponding anatomical axis of anatomical joints of the user's upper extremity for each anatomical rotation of segments of the user's upper extremity in anatomical joints;
a securement of said exoskeleton structure to upper extremity segments in such a way where said disposition of said rotation axes of said modules is preserved during whole cycle of the predetermined locomotor act;
an adjustment selectively of an exercise load in each said resistance mechanism of said exoskeleton structure in accordance with the predetermined locomotor act;
a performing of the predetermined locomotor act by the user;
a measurement of muscle forces and joint angles of the user's upper extremity by said force sensors, and said angle sensors of said exoskeleton structure.

5. A method to claim 4,
wherein the anatomical joints of the user's upper extremity that being loaded and measured by said exoskeleton structure are a sternoclavicular joint with its two anatomical rotations, a shoulder joint with its three anatomical rotations, an elbow joint with its two anatomical rotations, and a wrist joint with its two anatomical rotations.
